(19) United States
(12) Patent Application Publication
(10) Pub. No.: US 2019/0086425 A1
(43) Pub. Date: Mar. 21, 2019

(54) Diagnostic for sjorgen's syndrome based on a biomarker

(71) Applicants: Earl Lee White, Iola, TX (US); Kevin Dawson, Pasadena, CA (US)

(72) Inventors: Earl Lee White, Iola, TX (US); Kevin Dawson, Pasadena, CA (US)

(21) Appl. No.: 15/732,255

(22) Filed: Oct. 13, 2017

Related U.S. Application Data
(60) Provisional application No. 62/496,341, filed on Oct. 14, 2016.

(51) Int. Cl. G01N 33/68; C07K 14/47; C07K 16/18

(52) U.S. Cl. CPC G01N 33/6893; C07K 14/47; G01N 2800/24; C07K 2317/34; C07K 16/18

(57) ABSTRACT

A method, assay, peptide and antibody that forms an antibody-antigen conjugate with the peptide comprising the amino acid sequence:

GPPPPPGKPQGPPPQGGNKPQGPPPPGKPQGPPAQGGSKSQSARAPPGKPQGPPQQE GNNPQGPPPPAGGNPQQPQAPP or an amino acid sequence that is at least 75% identical to said sequence, and the formation of an antigen-antibody conjugate is a positive indictor for Sjögren's Syndrome.

### CROSS-REFERENCE TO RELATED APPLICATION

This application claims priority to U.S. Provisional Application Ser. No. 62/496,341 filed Oct. 14, 2016, the disclosure of which is incorporated herein by reference in their entirety.

### REFERENCE TO SEQUENCE LISTING

The content of the submitted sequence listing on compact disc is in ASCII format (“TG-ST25 SeqListing”, The file is a sequence Listing 4,733 bytes, created on Oct. 8, 2017) filed with the application is incorporated herein by reference in its entirety.

### FIELD OF THE INVENTION

This invention is related to the field of disease diagnostics, and particularly to the field of diagnosing autoimmune diseases and more specifically to diagnosing Sjögren's Syndrome (SjS).

### BACKGROUND OF THE INVENTION

SjS is a systemic inflammatory disease affecting primarily the lacrimal and salivary glands [1, 2]. It may exist as a primary disorder (primary SjS) or can be associated with other autoimmune diseases (secondary SjS), for example, rheumatoid arthritis, systemic lupus erythematosus or systemic sclerosis. The prevalence of this syndrome varies widely depending on the criteria for classification, but it is estimated that between 1 and 3 million North Americans suffer from this disease [3, 4]. Clinically, if unmanaged, SjS can have a severe impact on the integrity of the oral cavity [1, 2]. SjS patients usually experience difficulty of swallowing, severe and progressive tooth decay, and oral infections (particularly fungal). To date, there are no known cures for this disease, however, treatment consists of symptomatic treatment and immunosuppressants. Since SjS affects both lacrimal and salivary glands, it is likely that tear and saliva could be used to extract a peptide that could be used for antibody generation. The same peptide may also be detected in other biological fluids, e.g., serum and urine.

Treatments include eye drops, medications, and eye surgery. Artificial tears moisten eyes to provide soothing relief. Medications include corticosteroids, saliva production stimulators, nonsteroidal anti-inflammatory drugs, and immunosuppressive drugs.

Corticosteroids modify or simulate hormonal effects, often to reduce inflammation, tissue growth, and repair. Common drugs include methylprednisolone (Medrol, Solu-Medrol, Hybrisil, A-Methapred, and Depo-Medrol) and loteprednol (Lotemax and Alrex).

Saliva production stimulator increases the production of saliva. Common drugs include cevimeline (Evoxac), pilocarpine (Salagen and Pilopine).

Nonsteroidal anti-inflammatory drugs relieve pain, decrease inflammation, and reduce fever. Common drugs include ibuprofen (Advil, Motrin, Midol, NeoProfen, Caldolor, Profen, Select, and Ibu), naproxen (Naprelan, Aleve, Naprosyn, Midol, Ec-Naprosyn, Rugby, Select, and Anaprox), and piroxicam (Feldene).

Immunosuppressive drugs reduce the immune response. Common drugs include rituximab (Rituxan) and hydroxychloroquine (Plaquenil).

SjS may be diagnosed by primary care physicians, specialists, including rheumatologists and ophthalmologist, and dentists.

SjS is considered to be the most under-diagnosed autoimmune disease. It is not uncommon for there to be a delay of 5 to 8 years after symptom onset before a diagnosis is made [5, 6]. Furthermore, the diagnostic approach to SjS is complicated because it must include two different goals: firstly, assessment of the ocular and salivary components, and secondly, differentiation between the primary and secondary variants of the syndrome [3]. Current diagnostic criteria for primary SjS include subjective (patient reported) and objective (measurable) signs of dry eye and/or dry mouth, presence of autoantibodies against the ribonucleoproteins Ro/SSA and/or La/SSB, and focal lymphocytic sialoadenitis in a salivary gland biopsy [3]. These criteria can only detect patients with established disease when glandular destruction has already occurred.

A previous study [7] determined that a biomarker did exist that can be used for diagnosis of SjS in saliva but required a lengthy sampling process followed by a data analysis process with an algorithm that must compare a disease group with a control group. This process identified one biomarker that was present exclusively in SjS patients' saliva as a degradation product of human basic salivary proline-rich protein 3 (PRP3) precursor (Scheme I).

SCHEME I
Amino acid sequence for salivary
proline-rich protein 3 (PRP3) precursor
(UniProtKB-Q04118 (PRB3_HUMAN)).
There are 309 amino acids in the sequence
with the first 16 being the signal peptide.
SEQ ID NO: 1
MLLILLSVAL LALSSAQSLN EDVSQEESPS VISGKPEGRR
PQGGNQPQRT PPPPGKPEGR PPQGGNQSQG PPPRPGKPEG
PPPQGGNQSQ GPPPRPGKPE GQPPQGGNQS QGPPPRPGKP
EGPPPQGGNQ SQGPPPRPGK PEGPPPQGGN QSQGPPPHPG
KPEGPPPQGG NQSQGPPPRP GKPEGPPPQG GNQSQGPPPR
PGKPEGPPPQ GGNQSQGPPP RPGKPEGSPS QGGNKPQGPP
PHPGKPQGPP PQEGNKPQRP PPPGRPQGPP PPGGNPQQPL
PPPAGKPQGP PPPPQGGRPH RPPQGQPPQ

In the previous study, 2 μL of saliva from each sample were diluted in 180 μL of 0.1% trifluoroacetic acid (TFA), co-crystalized with MALDI matrix solution containing 10 mg/mL α-cyano-4-hydroycinnamic acid and analyzed by MALDI-TOF MS. The raw mass spectral data were analyzed using PROFILE™, a proprietary biomarker discovery platform optimized for the sensitive and accurate identification of disease-specific proteomics signatures in high-resolution MALDI-TOF mass spectra. Saliva sample proteins and peptides were fractionated by high performance liquid chromatography (HPLC) prior to protein sequencing by Edman degradation. Mass spectra of each fraction was acquired and data interrogated for detection of candidate biomarkers. An aliquot of the remainder of each fraction was subjected to Edman sequencing on an Applied Biosystems 494HT instrument.

Saliva samples from control subjects and SjS patients were processed for mass spectrometry analyses. One SjS-specific marker (m/z=3803.38) showed peaks with 0.5 amu periods in the m/z=3,803-3,808 range. As expected, another SjS-specific marker was detected in the m/z=7,606-7,618 range with 1.0 amu periods, which confirms that the SjS-specific marker at m/z=3,803.38 is a doubly charged ion. This discovery provides not only a novel putative biomarker for SjS but also an unexpected technical advantage. Namely, the doubly charged ion in the MALDI-TOF spectra can be detected at high sensitivity because it is located between the background peaks where signals from singly charged ions were not detected. Noteworthy is that this biomarker was detected in both primary and secondary SjS patients.

Experiments were conducted to further characterize the molecular identity of the doubly charged ion that was only present in SjS patients' saliva samples (FIG. 1). Fractions containing the peptide associated with the doubly charged ion were concentrated and subjected to Edman sequencing. A total of forty amino acids were positively identified using Edman degradation sequencing (N-terminus to C-terminus) SPPA KPQG PPPQ GGNQ PQGP PPPP GKPQ GPPP QGGN KPQG SEQ ID NO:2. The partial amino acid sequence was searched against the non-redundant (nr) GenBank database and was determined to be a good match to the human basic salivary proline-rich protein 3 precursor.

The first ten amino acids of the 7,606 Da biomarker peptide, SPPAKPQGPP SEQ ID NO:3, conform to a furin recognition sequence (XPPXXPXXP) SEQ ID NO:4 which was shown to be present in precursor PRPs and to be cleaved by furin, in vitro and in vivo [13-15]. It is therefore likely that this biomarker peptide is a proteolytic product from one of the salivary PRPs. Furthermore, the fact that this peptide was only present in saliva from SjS patients, suggests that the activity of the convertase(s) responsible for its generation is high in SjS patients' salivary glands.

Saliva has been extensively used to aid in the diagnosis of several diseases including periodontal diseases, salivary gland diseases and dysfunctions, viral diseases, sarcoidosis, tuberculosis, lymphoma, gastric ulcers, liver dysfunction, and cancer [8, 9-12].

Saliva samples were collected from SjS patients and healthy controls to be used for detecting peptides. Samples from healthy subjects have been shown to not contain the peptide of interest. Since this peptide fragment is present as part of the full protein in healthy patients, it remains unknown whether an antibody against the fragment does not cross-react with the full protein thus producing false positives for the disease.

### SUMMARY

The present invention is directed to a peptide, antibody, antibody-antigen conjugate, and a method of testing for SjS and to a diagnostic test.

In an embodiment of the invention, a peptide indicative of SjS is determined and isolated.

In another embodiment, an antibody is generated. The antibody binds specifically to the peptide that only occurs in SjS patients. The antibody can be a monoclonal antibody, a polyclonal antibody (whether purified or antisera), recombinant antibody, antibody fragment or a set of antibodies that bind specifically to a SjS specific peptide.

In another embodiment, a test for SjS is developed by detecting an antibody-antigen conjugate between an antibody that specifically binds to an SjS peptide and the peptide.

In another embodiment, a method of testing is identified that utilizes an antibody that binds to a SjS peptide and the conjugate that is formed can be observed by any of a number of known methods.

In another embodiment, a diagnostic test for SjS utilizes an antibody that binds to a SjS peptide. The test may be comprised of the antibody, and a coloring agent on a paper test strip; an antibody in a vial where precipitation of the antibody-antigen conjugate can be observed or any other known immunoassay format.

### BRIEF DESCRIPTION OF THE DRAWING:

FIG. 1. MALDI-TOF mass spectra (A) heat map representation (B) doubly-charged peaks at m/z=3803.4 and (C) protonated molecular ion at m/z 7606.8 for saliva of confirmed Sjögren's Syndrome patient.

### DETAILED DESCRIPTION OF THE INVENTION

Development of a diagnostic test for Sjögren's Syndrome (SjS) starts with collection of saliva to isolate and capture an active form of the peptide or protein fragment responsible for the SjS specific peptide sequence with MW of 7606 Da

GPPPPPGKPQGPPPQGGNKPQGPPPPGKPQGPPAQGGSKSQSARAP
PGKPQGPPQQEGNNPQGPPPPAGGNPQQPQAPP

SEQ ID NO:5 followed by the generation of antibodies against this peptide/protein fragment. Once the antibodies have been generated, the next step is testing the antibody with saliva from patients with confirmed SjS relative to normal control saliva samples. Any conventional immunoassay format may be used. From this point on, the form of a test could be as simple as a test strip impregnated with the antibody to a solution containing the antibody that would react with the peptide in patient saliva samples or other biological fluids being detected by a color change or a colorimetric device that could yield quantitative results about the disease. An active form of the peptide or protein fragment is then synthesized and tested.

Antibodies are generated to the active form of the peptide/protein fragment responsible for the sequence with MW of 7606 Da. Typically, this is done by exposing rabbits to the peptide/protein fragment and harvesting serum antibodies once the rabbit develops an immune response.

Since this peptide/protein fragment is present in the full protein of healthy patients, a discriminating antibody must specifically bind the fragment but not react with the full protein. Antibodies that bind to the full protein would produce false positives for the disease. Once the antibody has been generated, the next step is testing the antibody with saliva from patients with confirmed SjS relative to normal control saliva samples.

In one embodiment, the antibodies bind specifically to the marker peptide/protein fragment responsible for the sequence with MW of 7606 Da and do not react to the full protein are placed on a solid phase such as a test strip along with other optional components either in the test strip or, mixed with the sample or separately added so that a detectable change (e.g. a change color) upon combination of the antibody with the peptide.

In another embodiment, other immunoassay formats may be used with different components bound to different solid phases or in solution and different detection schemes used. However, in each format, the critical step is the specific binding of the antibody of the present invention to the peptide of the invention occurs.

A saliva sample or other body fluid (e.g. tears, nasal secretions, etc.) is collected from a patient. This sample serves as the peptide containing liquid. The sample may be fractionated to at least partially purify the sample or to remove any potentially interfering substance(s). The antibody specific to the SJS peptide is exposed to the sample. Binding is typically detected by a positive color change that indicates whether or not the saliva sample contains a peptide as a marker specific for SJS.

In another embodiment, the antibody-peptide binding is quantified. In such a situation, typically, the signal from a labeled antibody or a labeled peptide is quantitatively measured. For example, the amount of a color change may be measured by a colorimetric device. Spectrophotometers serve well for this purpose. Some other common diagnostic tests that can be developed include a multi-strip test that can be used to test multiple samples simultaneously. This strip could be a device with six or twelve wells coated with antibodies and an indicator that changes color when reacted with an antigen. This type of a device, similar to the diagram shown below, could be used to perform replicate analyses of a single sample or different biological samples or different concentrations of either sample or reagents to either quantify the results and/or to perform a confirmation.

### Types of Antibody-Antigen Reactions That May be Used in This Invention and to Build a Test.

Precipitation reaction. When an antigen-antibody reaction takes place, the antigens and antibodies cross-link to form a lattice-like structure that precipitates out of the solution, settling onto the bottom of a vial. Observation of this precipitate can confirm the presence of a specific peptide indicative of SjS. To enhance effectiveness, plural antibodies binding to different portions of the peptide may be used. Also, a secondary antibody or other agent such as Protein A may be added that binds to the antibody to enhance precipitation. In a competitive type assay, the lack of a precipitate can confirm the presence of the SjS specific peptide in the sample.

Agglutination reaction. Agglutination refers to a clumping that occurs when an antigen comes into contact with its corresponding antibody. Observation of an agglutination reaction (or lack of agglutination) aids in the detection of a specific peptide, such as those produced in SjS.

Complement fixation refers to a reaction in which an antigen binds with an antibody, forming a combination that causes complement to become fixed at the same site. Detection of this complement fixation reaction leads to identification of the peptide produced by a SjS patient.

Immunofluorescent assay is a technique in which specific antibodies are tagged with a fluorescent dye. When these antibodies bind to the SjS indicator peptide, they appear as fluorescent, glowing particles under a fluorescent microscope, or are measured in a fluorescent spectrometer, thereby revealing their location, particularly when bound to the peptide as an indicator of the peptide being present in the sample. Likewise, the peptide may be tagged with a fluorescent dye for the opposite effect. The tagged peptide may be either the sample peptide or a previously synthesized peptide.

Enzyme immunoassays uses tagging with an enzyme. An enzyme substrate or product is a readily detectable substance which measurement of the formation or reduction of the readily detectable substances determines the presence of the antibody or peptide tagged. Typically, a developing solution is added that the enzyme catalyzes to form a color change. Thus a simple viewing of the color determine whether or not the sample comes from a patient with SkS. Of particular interest is an Enzyme-linked immunosorbent assay (ELISA). In this format the antibody is first bound to a solid phase such as the inside of a container or on a small particle. The sample containing a peptide is added and allowed to bind to the antibody. A tagged second specific antibody is then added which binds to the peptide so that the peptide is “sandwiched” between the antibodies. Optionally, a developing solution is added for the tag to react with and the result is measured or observed. Presence of the readily detectable signal indicates presence of the peptide in the sample. Alternatively, the binding of antibody to solid phase may be performed later in the assay and the antibody-peptide binding may be performed before adding it to a container.

Radioimmunoassay (RIA) is a similar technique to an immunofluorescent assay that tags antibody or peptide with radioactive material. Detection is typically performed by a scintillation counter.

Some of these tests can be incorporated into a diagnostic test.

In another embodiment of the present invention, the SjS specific peptide of the present invention may be isolated from patient fluid samples but is preferably chemically synthesized to form a pure chemical compound. Because of natural polymorphisms and mutations, variations in the amino acid sequence may be present. Furthermore, as the peptide is presumed to be a degradation product, a slightly truncated or lengthened peptide is possible In as much as only small portions of the entire sequence serves as antibody binding site(s), such changes may be made to the sequence as still be encompassed by the present invention, provided that SjS specific reactivity remains. Up to about 25% variation in the amino acid sequence is acceptable provided that at least one SjS specific antibody can be made. Of particular interest is a truncated peptide containing the 57 amino acids at the carboxy-terminal of SEQ ID NO:5.

The SjS specific peptide of the present invention may be chemically coupled to itself or even polymerized to form a molecule with multiple identical antibody binding sites. This has certain advantages with some immunoassay formats. Such techniques are known per se.

The SjS specific peptide of the present invention may be chemically coupled to a diverse protein or peptide that is strongly antigenic (such as KLH, albumin, etc.) so as to elicit a strong immune response when used to immune an animal for the purposes of producing antibodies.

The SjS specific peptide defined by SEQ ID NO:1 and antibody that specifically binds to it may separately be chemically coupled to a tag for the purposes of producing a reagent for an immunoassay to detect SjS. A large number of tags, known per se, have been used in various different immunoassays such as fluorescent, radioactive or enzyme molecules or solid phase particles.

The term “antibody” is used to broadly define a specific binding partner. While conventional polyclonal antibodies are typically used, monospecific antibodies, monoclonal antibodies, antibody fragments, recombinant molecules such as single-chain “antibodies” (scFv) and other specific binding molecules such as those determined by panning a library of molecules, or the natural biological receptor (or portions thereof) that are naturally bound by the SjS specific peptide of the present invention and the like. In all situations, the “antibody” should bind with high affinity and with sufficient specificity to distinguish SjS samples from non-SjS samples.

The assay of the present invention may be in the form of a kit. Such a kit contains at least a container containing an antibody specific for the SjS specific peptide of the present invention and instruction on using it in an immunoassay. Tagged antibody or tagged peptide is preferably included. Developing reagents such as enzyme substrates and apparatus such as a multi-welled plate may also be present.

The assay of the present invention may be used in conjunction with other assays for SjS to make a combination method and combination kit containing reagents for plural SjS assays based on different peptides/proteins. For example anti-nuclear antigen and rheumatoid factor are known to be associated with SjS. Others have also been proposed such as Maślińska et al, Reumatologia. 2017; 55 (3):113-119, Tzartos et al, Rheumatology (Oxford) 2017 Aug. 31, Nezos et al, Clin Immunol. 2017 Sep. 14 and Zoukhri et al, Journal of Oral Science Vol. 54 (2012) No. 1 March P 61-70. Any of these and/or other assays may be included in the combination kit along with the reagents used for detecting the SjS specific peptide of the invention in a sample.

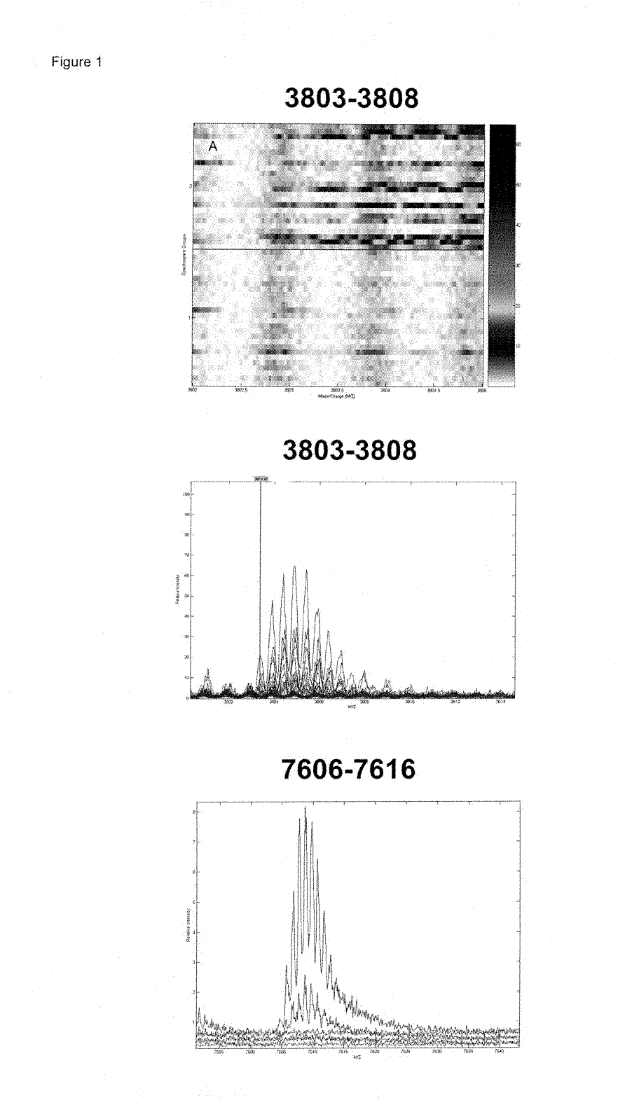

Example 1: Saliva samples from known SjS patients and control humans were taken by suction. Two μl of salvia were processed for mass spectrometry analyses on a prOTOF 2000 matrix-assisted laser desorption/ionization orthogonal time of flight (MALDI O-TOF) mass spectrometer. Spectral data obtained for 750-12000 daltons is shown in FIG. 1. The results were highly reproducible and contained many peptides and peptide fragments in the 750-7,500 Da range.

Bioinformatic data analysis using spectral data were analyzed using PROFILE™, a proprietary biomarker discovery platform optimized for signatures in high-resolution MALDI-TOF mass spectra. The bioinformatics have been previously used for other biomarker discovery; for example, Lopez et al, (2005) Clin Chem 51, 1946-1954, Avasarala et al (2005) J Mol Neurosci 25, 119-125 and Brouwers et al (2005) Endocr Relat Cancer 12, 263-272. The saliva samples were fractionated by HPLC and then peptides sequenced by Edman degradation. By comparison of SjS and normal samples, the SjS specific peptide sequence with MW of 7606 Da having the SEQ ID NO: 5 was determined and found only in SjS samples and not in normal samples.

Example 2: An antibody is made by injecting the peptide from Example 1 that indicates SjS into an animal. After 6 weeks, blood is withdrawn and serum collected. This polyclonal antibody containing antisera is used for detecting the SjS specific peptide. The antibody is absorbed onto the inside of wells in a 96 well plate. Albumin is later adsorbed on the inside of the same wells. The plate is washed in phosphate buffered saline (PBS). Saliva samples from both normal and SJS patients are added to each well in duplicate and at 10 fold and 100 fold dilutions. After allowing to incubate for 15 minutes at 37 degrees C., the plate is emptied and rinsed with additional PBS. Antibody previously tagged with enzyme is added and again incubated for 15 minutes at 37 degrees C. and rinsed in PBS. Enzyme substrate is added and the color change (clear to blue) observed visually and quantitative measured in a standard Microtiter plate reader. Samples from SjS patients are noticeably different.

Example 2: Another test similar to Example 1 is made using tiny polystyrene beads as the solid phase instead of the 96 well plate. Albumin-blocked 96 well plates are used as vessels for the binding and developing reactions. Filter containing pipette tips are used for aspirating liquid and beads in lieu of emptying . The tagged antibody component is tagged with fluorescein isothiocyanate. The conditions of the method of Example 1 is repeated except for a final pipetting beads on a glass slide and visualizing under ultraviolet light instead of adding enzyme substrate. The results are seen visually (and optionally may be measured quantitatively

It will be understood by a person skilled in the art that a number of antibodies and similar molecules are within the scope and spirit of the invention; that many known assays can be adapted to the diagnosis of SjS and that any diagnostic kit that is not specifically recited herein are within the spirit and intent of this invention. All references cited herein are incorporated by reference.

### REFERENCES

Vitali C, Bombardieri S, Jonsson R, Moutsopoulos H M, Alexander E L, Carsons S E, Daniels T E, Fox P C, Fox R I, Kassan S S, Pillemer S R, Talal N, Weisman M H. Classification criteria for Sjögren's syndrome: a revised version of the European criteria proposed by the American-European Consensus Group. Ann Rheum Dis 2002; 61:554-558.

Fox R I. Pathogenesis of Sjögren's syndrome. Rheum. Dis. Clin. North Am. 1992; 18:517-538

Coll J, Rives A, Grino M C, Setoain J, Vivancos J, Balcells A. Prevalence of Sjogren's syndrome in autoimmune diseases. Ann Rheum Dis 1987; 46:286-289.

Schaumberg D A, Sullivan D A, Buring J E, Dana M R. Prevalence of dry eye syndrome among US women. Am J Ophthalmol 2003; 136:318-326.

Daniels T E. Sjogren's syndrome: clinical spectrum and current diagnostic controversies. Adv Dent Res 1996; 10:3-8.

Asmussen K. Monitoring the disease activity. Scand J Rheumatol Suppl 2001: 23-26.

Zoukhri, D, Rawe I, Singh, M, Kublin, C, Dawson, K, Haddon, W, White, E, Hanley, K, Tusé, D, Malyj, W and Papas, A. Discovery of Putative Salivary Biomarkers for Sjögren's Syndrome Using High Resolution Mass Spectrometry and Bioinformatics. J. of Oral Sci. 2012: 54-1: 61-70.

Kaufman E, Lamster I B. The diagnostic applications of saliva—a review. Crit Rev Oral Biol Med 2002; 13:197-212.

Balicki R, Grabowska S Z, Citko A. Salivary epidermal growth factor in oral cavity cancer. Oral Oncol 2005; 41:48-55.

Dziemianczyk D, Grabowska S Z, Balicki R. Evaluation of secretory mucin concentration of patients with squamous cell carcinoma oral cavity. Rocz Akad Med Bialymst 2005; 50:334-338.

Sistig S, Lukac J, Vucicevic-Boras V, Delic D, Kusic Z. Salivary immunoglobulin A and G subclasses in HIV positive patients. Eur J Med Res 2003; 8:543-548.

Tabak L, Mandel I D, Karlan D, Baurmash H. Alterations in lactoferrin in salivary gland disease. J Dent Res 1978; 57:43-47.

Cai K, Bennick A. Processing of acidic proline-rich proprotein by human salivary gland convertase. Arch Oral Biol 2004; 49:871-879.

Stubbs M, Chan J, Kwan A, So J, Barchynsky U, Rassouli-Rahsti M, Robinson R, Bennick A. Encoding of human basic and glycosylated proline-rich proteins by the PRB gene complex and proteolytic processing of their precursor proteins. Arch Oral Biol 1998; 43:753-770.

Chan M, Bennick A. Proteolytic processing of a human salivary proline-rich protein precursor by proprotein convertases. Eur J Biochem 2001; 268:3423-3431. A person skilled in the are of making diagnostic tests would be aware that the several types of test that use an antibody would be within the scope of this patent.

We claim:

1. A peptide comprising the amino acid sequence:
GPPPPPGKPQGPPPQGGNKPQGPPPPGKPQGPPAQGGSKSQSARAPPGKPQGPPQQ EGNNPQGPPPPAGGNPQQPQAPP SEQ ID NO: 5 or an amino acid sequence that is at least 75% identical, and is a biomarker for Sjögren's Syndrome.

2. The peptide of claim 1 chemically bound to a strongly antigen protein and capable of eliciting an immune response in an animal.

3. The peptide of claim 1 chemically bound to a tag.

4. The peptide of claim 1 having the carboxy-terminal 57 amino acids of SEQ ID NO: 5.

5. An antibody that specifically binds to and forms an antibody-antigen conjugate with a peptide comprising the amino acid sequence:
GPPPPPGKPQGPPPQGGNKPQGPPPPGKPQGPPAQGGSKSQSARAPPGKPQGPPQQ EGNNPQGPPPPAGGNPQQPQAPP SEQ ID NO: 5 or an amino acid sequence that is at least 75% identical and is a biomarker for Sjögren's Syndrome.

6. The antibody of claim 5 chemically bound to a tag.

7. The antibody of claim 5 that binds to the carboxy-terminal 57 amino acids of SEQ ID NO: 5.

8. The antibody of claim 5 wherein the antibody is selected from the group consisting of a polyclonal antibody, a monoclonal antibody, a recombinant antibody, a specific binding protein or peptide, and fragments of each.

9. A method for detecting a biomarker for Sjögren's Syndrome comprising;
obtaining a body fluid sample from a patient suspected of having Sjögren's Syndrome, incubating proteins or peptides in the sample with an antibody specifically binding a peptide comprising the amino acid sequence:
GPPPPPGKPQGPPPQGGNKPQGPPPPGKPQGPPAQGGSKSQSARAPPGKPQGPPQQ EGNNPQGPPPPAGGNPQQPQAPP SEQ ID NO: 5 or an amino acid sequence that is at least 75% identical, and is a biomarker for Sjögren's Syndrome, and detecting the presence of binding between the antibody and the peptide.

10. The method of claim 9 further comprising;
Adding a second antibody specifically binding the peptide, and
Wherein binding between the peptide to both the antibody and the second antibody is detected.

11. The method of claim 9 wherein the antibody is tagged.

12. The method of claim 11 further comprising adding at least one reagent that interacts with the tag to produce a readily detectable signal.

13. The method of claim 12 wherein the readily detectable signal is measured quantitatively.

14. The method of claim 11 wherein the tag is a radioactive atom, a fluorescent molecule, an enzyme, or an insoluble solid phase.

15. The method of claim 9 further comprising adding a tagged peptide comprising the amino acid sequence:
GPPPPPGKPQGPPPQGGNKPQGPPPPGKPQGPPAQGGSKSQSARAPPGKPQGPPQQE GNNPQGPPPPAGGNPQQPQAPP SEQ ID NO: 5 or an amino acid sequence that is at least 75% identical.

16. The method of claim 15 wherein the tagged peptide is incubated with and binds to the antibody before the sample contacts the antibody.

17. A kit for performing the method of claim 9 comprising a container containing the antibody and instructions for its use to detect Sjögren's Syndrome.

18. The kit of claim 17 wherein the antibody is tagged.

19. The kit of claim 18 further comprising a container of liquid reagent capable of interacting with the tag to generate a readily detectable signal.


SEQUENCE LISTING (5 sequences)

SEQ ID NO: 1 - protein, 309 aa, HOMO SAPIENS
MLLILLSVALLALSSAQSLNEDVSQEESPSVISGKPEGRRPQGGNQPQRTPPPPGKPEGRPPQGGNQSQGPPPRPGKPEGPPPQGGNQSQGPPPRPGKPEGQPPQGGNQSQGPPPRPGKPEGPPPQGGNQSQGPPPRPGKPEGPPPQGGNQSQGPPPHPGKPEGPPPQGGNQSQGPPPRPGKPEGPPPQGGNQSQGPPPRPGKPEGPPPQGGNQSQGPPPRPGKPEGSPSQGGNKPQGPPPHPGKPQGPPPQEGNKPQRPPPPGRPQGPPPPGGNPQQPLPPPAGKPQGPPPPPQGGRPHRPPQGQPPQ

SEQ ID NO: 2 - protein, 40 aa, HOMO SAPIENS
SPPAKPQGPPPQGGNQPQGPPPPPGKPQGPPPQGGNKPQG

SEQ ID NO: 3 - protein, 10 aa, HOMO SAPIENS
SPPAKPQGPP

SEQ ID NO: 4 - protein, 10 aa, HOMO SAPIENS
SPPAKPQGPP

SEQ ID NO: 5 - protein, 79 aa, HOMO SAPIENS
GPPPPPGKPQGPPPQGGNKPQGPPPPGKPQGPPAQGGSKSQSARAPPGKPQGPPQQEGNNPQGPPPPAGGNPQQPQAPP